(19) United States
(12) Patent Application Publication
(10) Pub. No.: US 2019/0223477 A1
(43) Pub. Date: Jul. 25, 2019

(54) METHODS OF INCREASING THE OMEGA-3 POLYUNSATURATED FATTY ACIDS LEVELS IN HUMAN PLASMA

(71) Applicant: DEVENISH NUTRITION LIMITED, Belfast, Antrim (GB)

(72) Inventor: Heather HAYES, Lisburn (GB)

(73) Assignee: Devenish Nutrition Limited, Belfast, Antrim (GB)

(21) Appl. No.: 16/314,350

(22) PCT Filed: Jun. 30, 2017
(86) PCT No.: PCT/EP2017/066404

§ 371 Date: Dec. 28, 2018

(30) Foreign Application Priority Data
Jun. 30, 2016 (GB) 1611503.2

(51) Int. Cl. A23L 13/40; A23L 33/00; A23K 10/16; A23K 10/30; A23K 20/158; A23L 33/12; A23K 50/75; A61K 31/202

(52) U.S. Cl. CPC A23L 13/43; A23L 33/30; A23K 10/16; A23K 10/30; A23V 2002/00; A23L 33/12; A23K 50/75; A61K 31/202; A23K 20/158

(57) ABSTRACT

The present invention relates to animal meat enriched with Omega-3 polyunsaturated fatty acids for use in increasing the Omega-3 polyunsaturated fatty acids levels in human plasma by dietary administration of the animal meat enriched with Omega-3 polyunsaturated fatty acids to a subject. Also disclosed is a method of increasing the Omega-3 polyunsaturated fatty acids levels in human plasma comprising dietary administration of the animal meat enriched with Omega-3 polyunsaturated fatty acids to the subject.

### FIELD OF THE INVENTION

The present invention relates to a composition comprising at least one source of Omega-3 polyunsaturated fatty acid, and use thereof in enriching animal meat with Omega-3 polyunsaturated fatty acids. Also disclosed are an animal feed comprising a composition of the invention, use each thereof in enriching animal meat with Omega-3 polyunsaturated fatty acids; methods for enriching animal meat with Omega-3 polyunsaturated fatty acids; and uses each thereof in increasing the Omega-3 polyunsaturated fatty acid levels in human plasma after consumption of this enriched animal meat and reducing the risk factors of cardiovascular disease.

### BACKGROUND TO THE INVENTION

Omega-3 fatty acids are polyunsaturated fatty acids (PUFAs) with a double bond (C═C) at the third carbon atom from the end of the carbon chain. The three types of omega-3 fatty acids involved in human physiology are a-linolenic acid (ALA, 18 carbon atoms and 3 double bonds C18:3 n3), eicosapentaenoic acid (EPA, 20 carbon atoms and 5 double bonds C20:5 n3), and docosahexaenoic acid (DHA, 22 carbon atoms and 6 double bonds C22:6 n3).

Omega-3 fatty acids are important for normal metabolism, but mammals are unable to synthesize omega-3 fatty acids in their bodies and must therefore consume them through diet. There is a very limited conversion of dietary ALA into the more important long-chain omega-3 fatty acids, EPA and DHA, in the body but this is insufficient to meet a mammal's needs and therefore all three fatty acids must be consumed in the diet.

The recommended daily allowance of EPA and DHA is >250 mg/day (European Food Safety Authority). Oily fish is the principal dietary source of EPA and DHA in the diet, and consumers are recommended to eat at least one serving of oily fish per week. However, uptake of these recommendations is poor due to limited availability, cost and distaste of oily fish, and concern about toxins in such fish including methylmercury, polychlorinated biphenyls, and dioxins.

Many people do not eat fish at all and therefore, worldwide, deficiencies are common. A survey carried out on behalf of the Food Standards Agency and the Department of Health shows that there is an EPA/DHA deficiency across all age groups based on low intake of oily fish.

Omega-3 fatty acid dietary supplements do not consistently provide the same benefits as oily fish. Potential explanations include additional supplements are not equivalent to a balanced healthy diet, poor adherence, too late commencement, and differences in bioavailability.

Flaxseed, flaxseed oil, and canola are commonly incorporated into poultry diets to produce omega-3 fatty acid-fortified eggs. These ingredients contain high amounts of a-linolenic acid (ALA) compared with other oil seeds. However, due to low efficiencies of such conversions in vivo, supplementing diets for laying hens with ALA rarely produces eggs containing the required levels of DHA or EPA.

Therefore, there is a need to provide alternative sources of omega-3 fatty acids in mammalian diets.

### SUMMARY OF THE INVENTION

According to a first aspect of the present invention there is provided a composition comprising at least one source of Omega-3 polyunsaturated fatty acid.

Optionally, the at least one Omega-3 polyunsaturated fatty acid source is a plant polyunsaturated fatty acid source. Further optionally, the at least one plant Omega-3 polyunsaturated fatty acid source is a plant polyunsaturated fatty acid.

Optionally, the plant Omega-3 polyunsaturated fatty acid source or plant Omega-3 polyunsaturated fatty acid is plant cells or from plant cells.

Optionally, the plant Omega-3 polyunsaturated fatty acid source or plant Omega-3 polyunsaturated fatty acid is plant oil or from plant oil.

Optionally, the at least one plant Omega-3 polyunsaturated fatty acid source is an algal polyunsaturated fatty acid source. Further optionally, the at least one plant Omega-3 polyunsaturated fatty acid source is an algal polyunsaturated fatty acid.

Optionally, the composition comprises at least 5% algal Omega-3 polyunsaturated fatty acid source or algal Omega-3 polyunsaturated fatty acid. Further optionally, the composition comprises 5%-60% algal Omega-3 polyunsaturated fatty acid source or algal Omega-3 polyunsaturated fatty acid. Still further optionally, the composition comprises 5%, optionally 6%, optionally 7%, optionally 8%, optionally 9%, optionally 10%, optionally 11%, optionally 12%, optionally 13%, optionally 14%, optionally 15%, optionally 16%, optionally 17%, optionally 18%, optionally 19%, optionally 20%, optionally 21%, optionally 22%, optionally 23%, optionally 24%, optionally 25%, optionally 26%, optionally 27%, optionally 28%, optionally 29%, still further optionally 30%, still further optionally 40%, still further optionally 50%, still further optionally 60% algal Omega-3 polyunsaturated fatty acid source or algal Omega-3 polyunsaturated fatty acid.

Optionally, the algal Omega-3 polyunsaturated fatty acid source or algal Omega-3 polyunsaturated fatty acid is algal cells or from algal cells. Optionally, the algal cells are selected from any one or more of Chlorella, Spirulina, Schizochytrium, Crypthecodinium, Arthrospira, Porphyridium, and Nannochloropsis. Optionally, the algal Omega-3 polyunsaturated fatty acid source or algal Omega-3 polyunsaturated fatty acid is any one or more of Chlorella, Spirulina, Schizochytrium, Crypthecodinium, Arthrospira, Porphyridium, and Nannochloropsis algal cells; or from any one or more of Chlorella, Spirulina, Schizochytrium, Crypthecodinium, Arthrospira, Porphyridium, and Nannochloropsis algal cells.

Optionally, the algal Omega-3 polyunsaturated fatty acid source or algal Omega-3 polyunsaturated fatty acid is dehydrated algal cells or from dehydrated algal cells. Optionally, the dehydrated algal cells are selected from any one or more of Chlorella, Spirulina, Schizochytrium, Crypthecodinium, Arthrospira, Porphyridium, and Nannochloropsis. Optionally, the algal Omega-3 polyunsaturated fatty acid source or algal Omega-3 polyunsaturated fatty acid is dehydrated Chlorella, Spirulina, Schizochytrium, Crypthecodinium, Arthrospira, Porphyridium, and/or Nannochloropsis algal cells; or from dehydrated from any one or more of Chlorella, Spirulina, Schizochytrium, Crypthecodinium, Arthrospira, Porphyridium, and/or Nannochloropsis algal cells.

Optionally, the algal Omega-3 polyunsaturated fatty acid source or algal Omega-3 polyunsaturated fatty acid is an algal oil or from an algal oil.

Optionally, the algal oil is a marine algal oil. Optionally, the algal oil is a marine algal oil from a microscopic marine alga. Optionally, the microscopic marine alga are selected from any one or more od Chlorella, Spirulina, Schizochytrium, Crypthecodinium, Arthrospira, Porphyridium, and Nannochloropsis. Alternatively, the algal oil is a marine algal oil from a macroscopic marine alga. Optionally or additionally, the algal oil is a marine algal oil from a multicellular marine alga. Optionally, the algal oil is a marine algal oil from a red, brown, green alga, or a combination each thereof.

Optionally, the composition comprises at least 0.5% (w/w) algal oil. Further optionally, the composition comprises 0.5-25% (w/w) algal oil. Still further optionally, the composition comprises 0.5%, optionally 1%, further optionally 2%, still further optionally 3%, optionally 4%, further optionally 5%, still further optionally 6%, still further optionally 7%, still further optionally 8%, still further 30 optionally 9%, still further optionally 10%, still further optionally 11%, still further optionally 12%, still further optionally 13%, still further optionally 14%, still further optionally 15%, still further optionally 20%, still further optionally 25% (w/w) algal oil.

Optionally or additionally, the at least one plant Omega-3 polyunsaturated fatty acid source is a linseed (Linum usitatissimum) polyunsaturated fatty acid source. Further optionally, the at least one plant Omega-3 polyunsaturated fatty acid source is a linseed (Linum usitatissimum) polyunsaturated fatty acid.

Optionally, the composition comprises at least 5% linseed Omega-3 polyunsaturated fatty acid source or linseed Omega-3 polyunsaturated fatty acid. Further optionally, the composition comprises 5%-80% linseed Omega-3 polyunsaturated fatty acid source or linseed Omega-3 polyunsaturated fatty acid. Still further optionally, the composition comprises 5% optionally 10%, further optionally 15%, still further optionally 20%, still further optionally 30%, still further optionally 40%, still further optionally 50%, still further optionally 60%, still further optionally 70%, still further optionally 80% linseed Omega-3 polyunsaturated fatty acid source or linseed Omega-3 polyunsaturated fatty acid.

Optionally, the linseed Omega-3 polyunsaturated fatty acid source or linseed Omega-3 polyunsaturated fatty acid is linseed or from linseed. Further optionally, the linseed Omega-3 polyunsaturated fatty acid source or linseed Omega-3 polyunsaturated fatty acid is milled or ground linseed, or from milled or ground linseed. Still further optionally, the linseed Omega-3 polyunsaturated fatty acid source or linseed Omega-3 polyunsaturated fatty acid is micronized linseed or from micronized linseed.

Optionally, the linseed Omega-3 polyunsaturated fatty acid source or linseed Omega-3 polyunsaturated fatty acid is a linseed oil or from a linseed oil.

Optionally, the composition comprises at least 0.5% (w/w) linseed oil. Further optionally, the composition comprises 0.5% -25% (w/w) linseed oil. Still further optionally, the composition comprises 0.5%, optionally 1%, further optionally 2%, still further optionally 3%, optionally 4%, further optionally 5%, still further optionally 6%, still further optionally 7%, still further optionally 8%, still 20 further optionally 9%, still further optionally 10%, still further optionally 11%, still further optionally 12%, still further optionally 13%, still further optionally 14%, still further optionally 15%, still further optionally 20%, still further optionally 25% linseed oil.

Optionally, the at least one source of Omega-3 polyunsaturated fatty acid is an algal polyunsaturated fatty acid source and a linseed polyunsaturated fatty acid source. Further optionally, the at least one source of Omega-3 polyunsaturated fatty acid is an algal polyunsaturated fatty acid and a linseed polyunsaturated fatty acid.

Optionally, the composition comprises at least 5% algal polyunsaturated fatty acid source or algal polyunsaturated fatty acid and up to 60% linseed polyunsaturated fatty acid source or linseed polyunsaturated fatty acid. Further optionally, the composition comprises 5%-60% algal polyunsaturated fatty acid source or algal polyunsaturated fatty acid and 5%-80% linseed polyunsaturated fatty acid source or linseed polyunsaturated fatty acid. Still further optionally, the composition comprises 5%, optionally 6%, optionally 7%, optionally 8%, optionally 9%, optionally 10%, optionally 11%, optionally 12%, optionally 13%, optionally 14%, optionally 15%, optionally 16%, optionally 17%, optionally 18%, optionally 19%, optionally 20%, optionally 21%, optionally 22%, optionally 23%, optionally 24%, optionally 25%, optionally 26%, optionally 27%, optionally 28%, optionally 29%, still further optionally 30%, still further optionally 40%, still further optionally 50%, still further optionally 60% algal Omega-3 polyunsaturated fatty acid source or algal polyunsaturated fatty acid; and 5%, optionally 10%, further optionally 15%, further optionally 20%, further optionally 30%, further optionally 40%, still further optionally 50%, still further optionally 60%, still further optionally 70%, still further optionally 80% linseed Omega-3 polyunsaturated fatty acid source or linseed polyunsaturated fatty acid.

Optionally, the plant oil is an algal oil and a linseed oil.

Optionally, the composition comprises at least 0.5% (w/w) algal oil and up to 25% (w/w) linseed oil. Further optionally, the composition comprises 0.5% -25% (w/w) algal oil and 0.5%-25% (w/w) linseed oil. Still further optionally, the composition comprises 0.5%, optionally 1%, further optionally 2%, further optionally 3%, further optionally 4%, further optionally 5%, further optionally 6%, further optionally 7%, further optionally 8%, further optionally 9%, further optionally 10%, further optionally 15%, further optionally 20%, further optionally 25% (w/w) algal oil; and 25%, optionally 20%, optionally 15%, further optionally 10%, still further optionally 9%, still further optionally 8%, further optionally 7%, further optionally 6%, still further optionally 5%, still further optionally 4%, still further optionally 3%, still further optionally 2%, still further optionally 1%, still further optionally 0.5% (w/w) linseed oil.

Optionally or alternatively, the composition comprises a plant oil, optionally an algal oil, and linseed.

Optionally, the composition comprises at least 0.5% (w/w) algal oil and up to 80% (w/w) linseed. Further optionally, the composition comprises 0.5% -25% (w/w) algal oil and 5%-80% (w/w) linseed. Still further optionally, the composition comprises 0.5%, optionally 1%, further optionally 2%, further optionally 3%, optionally 4%, further optionally 5%, further optionally 6%, further optionally 7%, further optionally 8%, further optionally 9%, further optionally 10%, further optionally 11%, further optionally 12%, still further optionally 13%, still further optionally 14%, still further optionally 15%, still further optionally 20%, still further optionally 25% (w/w) algal oil; and 5%, optionally 6%, optionally 7%, optionally 8%, optionally 9%, optionally 10%, optionally 11%, optionally 12%, optionally 13%, optionally 14%, optionally 15%, optionally 16%, optionally 17%, optionally 18%, optionally 19%, optionally 20%, optionally 21%, optionally 22%, optionally 23%, optionally 24%, optionally 25%, optionally 26%, optionally 27%, optionally 28%, optionally 29%, still further optionally 30%, still further optionally 40%, still further optionally 50%, still further optionally 60% algal oil; and 5%, optionally 10%, further optionally 15%, further optionally 20%, further optionally 30%, further optionally 40%, still further optionally 50%, still further optionally 60%, still further optionally 70%, still further optionally 80% linseed.

Optionally, the linseed is milled or ground linseed. Further optionally, the linseed is micronized linseed.

Optionally, the composition consists of at least one plant polyunsaturated fatty acid source. Further optionally, the composition consists of at least one plant polyunsaturated fatty acid. Optionally, the plant polyunsaturated fatty acid source or plant polyunsaturated fatty acid is plant cells or from plant cells. Optionally, the plant polyunsaturated fatty acid source or plant polyunsaturated fatty acid is plant oil or from plant oil.

Optionally, the composition excludes a polyunsaturated fatty acid source from any of meat, fish, eggs, squid, and krill. Further optionally, the composition excludes polyunsaturated fatty acid from any of meat, fish, eggs, squid, and krill. Optionally, the Omega-3 polyunsaturated fatty acid source or Omega-3 polyunsaturated fatty acid excludes meat, fish, eggs, squid, and krill cells or is not from meat, fish, eggs, squid, or krill cells. Optionally, the Omega-3 polyunsaturated fatty acid source or Omega-3 polyunsaturated fatty acid excludes meat, fish, eggs, squid, and krill oil or is not from meat, fish, eggs, squid, or krill oil.

Alternatively or additionally, the composition comprises at least one fish polyunsaturated fatty acid source. Further optionally, the composition comprises at least one fish polyunsaturated fatty acid.

Optionally, the composition further comprises at least 5% fish Omega-3 polyunsaturated fatty acid. Further optionally, the composition further comprises 5% -60% fish Omega-3 polyunsaturated fatty acid. Still further optionally, the composition further comprises 5%, optionally 10%, further optionally 20%, further optionally 30%, still further optionally 40%, still further optionally 50%, still further optionally 60% fish Omega-3 polyunsaturated fatty acid.

Optionally, the fish Omega-3 polyunsaturated fatty acid source or fish Omega-3 polyunsaturated fatty acid is fish cells or from fish cells. Optionally, the fish cells are selected from sardine, herring, anchovy, salmon, trout, tuna, mackerel, cod liver, and krill. Optionally, the fish Omega-3 polyunsaturated fatty acid source or fish Omega-3 polyunsaturated fatty acid is sardine, herring, anchovy, salmon, trout, tuna, mackerel, cod liver, or krill cells; or from sardine, herring, anchovy, salmon, trout, tuna, mackerel, cod liver, or krill cells.

Optionally, the fish Omega-3 polyunsaturated fatty acid source or fish Omega-3 polyunsaturated fatty acid is a fish oil or from a fish oil.

Optionally, the at least one fish oil is encapsulated. Further optionally, the at least one fish oil is encapsulated with gelatine, cellulose or starch.

Optionally, the composition further comprises at least 0.5% (w/w) fish oil. Further optionally, the composition further comprises 0.5% -50% (w/w) fish oil. Still further optionally, the composition further comprises 0.5%, optionally 1%, optionally 2%, optionally 3%, optionally 4%, optionally 5%, optionally 6%, optionally 7%, optionally 8%, optionally 9%, optionally 10%, optionally 11%, optionally 12%, optionally 13%, optionally 14%, optionally 15%, optionally 16%, optionally 17%, optionally 18%, optionally 19%, further optionally 20%, further optionally 25%, optionally 30%, optionally 35%, still further optionally 40%, optionally 45%, still further optionally 50% (w/w) fish oil. Optionally, the at least one source of Omega-3 polyunsaturated fatty acid is an algal polyunsaturated fatty acid source, a linseed polyunsaturated fatty acid source, and a fish polyunsaturated fatty acid source. Further optionally, the at least one source of Omega-3 polyunsaturated fatty acid is an algal polyunsaturated fatty acid, a linseed polyunsaturated fatty acid, and a fish polyunsaturated fatty acid.

Optionally, the at least one source of Omega-3 polyunsaturated fatty acid is algal cells or from algal cells, linseed or from linseed, and fish cells or from fish cells. Optionally, the at least one source of Omega-3 polyunsaturated fatty acid is algal oil or from algal oil, linseed or from linseed, and fish cells or from fish cells. Optionally, the at least one source of Omega-3 polyunsaturated fatty acid is algal oil or from algal oil, linseed or from linseed, and fish oil or from fish oil. Optionally, the at least one source of Omega-3 polyunsaturated fatty acid is algal cells or from algal cells, linseed or from linseed, and fish oil or from fish oil.

Optionally, the composition comprises at least one plant oil, optionally an algal oil; linseed; and fish oil.

Optionally or additionally, the composition further comprises an antioxidant. Optionally, the composition further comprises at least 0.5% (w/w) antioxidant. Further optionally, the composition further comprises 0.5-5.0% (w/w) antioxidant. Still further optionally, the composition further comprises 0.5%, optionally 1.0%, further optionally 1.5%, still further optionally 2%, still further optionally 2.5%, still further optionally 3%, still further optionally 3.5%, still further optionally 4%, still further optionally 4.5%,still further optionally 5.0% antioxidant.

Optionally, the antioxidant is a naturally occurring antioxidant. Optionally, the antioxidant is selected from ascorbic acid, sodium ascorbate, calcium ascorbate, ascorbyl palmitate, tocopherol extracts from vegetable oils, tocopherol-rich extracts from vegetable oils, alpha-tocopherol, plant polyphenols, essential oils, and combinations each thereof. Further optionally, the composition further comprises 0.5-5% (w/w) naturally occurring antioxidant.

Optionally or additionally, the antioxidant is a synthetic antioxidant. Optionally, the antioxidant is selected from butylated hydroxyl toluene, butylated hydrox anisole, and combinations each thereof. Further optionally, the composition further comprises 0.5-2.5% (w/w) synthetic antioxidant.

Optionally or additionally, the antioxidant is combination of a naturally occurring antioxidant and a synthetic antioxidant.

According to a second aspect of the present invention there is also provided an animal feed comprising a composition according to the first aspect of the invention.

Optionally, the animal feed comprises 2.5-20% (w/w) of the composition. Further optionally, the animal feed comprises 2.5%, optionally 5.0%, further optionally 7.5%, still further optionally 10.0%, still further optionally 12.5%, still further optionally 15.0%, still further optionally 17.5%, still further optionally 20% (w/w) of the composition.

According to a third aspect of the present invention there is provided a composition according to the first aspect of the invention or an animal feed according to the second aspect of the invention for use in enriching animal meat with Omega-3 polyunsaturated fatty acids.

Optionally, the use comprises administration of the composition or the animal feed to an animal.

Optionally, the use comprises oral administration of the composition or the animal feed to an animal.

Optionally, the use comprises dietary administration of the composition or the animal feed to an animal.

15 Optionally, the use comprises dietary administration of the composition or the animal feed to an animal, wherein the composition amounts to 2.5-20% (w/w) of the animal feed or diet of the animal.

Optionally, the use comprises dietary administration of the composition or the animal feed to an animal, wherein the composition amounts to 2.5%, optionally 5.0%, further optionally 7.5.0%, still further optionally 10%, still further optionally 12.5.0% still further optionally 15.0%, still further optionally 17.5%, still further optionally 20% (w/w) of the animal feed or diet of the animal.

Optionally, the Omega-3 polyunsaturated fatty acids are selected from C12:1(n-3)cis cis-9-Dodecenoic acid, C18:3(n-3)cis Alpha-Linolenic acid (ALA), C18:4(n-3)cis Stearidonic acid, C20:3(n-3)cis cis-11,14,17-Eicosatrienoic acid, C20:4(n-3)cis cis-8,11,14,17-Eicosatetraenoic acid, C20:5(n-3)cis Eicosapentenoic acid (EPA), C22:5(n-3)cis Docosapentaenoic (DPA), Docosapentaenoic acid, C22:6(n-3)cis Docosahexaenoic (DHA)

Further optionally, the Omega-3 polyunsaturated fatty acids are selected from a-linolenic acid (ALA), eicosapentaenoic acid (EPA), docosahexaenoic acid (DHA), and combinations each thereof.

Optionally, the Omega-3 polyunsaturated fatty acids are a-linolenic acid (ALA).

Alternatively or additionally, the Omega-3 polyunsaturated fatty acids are docosahexaenoic acid (DHA).

Further alternatively or additionally, the Omega-3 polyunsaturated fatty acids are eicosapentaenoic acid (EPA).

Still further alternatively or additionally, the Omega-3 polyunsaturated fatty acids are selected from a combination of eicosapentaenoic acid (EPA) and docosahexaenoic acid (DHA) and are a-linolenic acid (ALA).

Optionally, the composition or method is for use in enriching animal meat with at least 40 mg Omega-3 polyunsaturated fatty acid per 100 g of animal meat. Further optionally, the composition or method is for use in enriching animal meat with 40-1500 mg Omega-3 polyunsaturated fatty acid per 100 g of animal meat. Still further optionally, the composition or method is for use in enriching animal meat with at least 50 mg, optionally at least 60 mg, further optionally at least 70 mg, still further optionally at least 80 mg, still further optionally at least 90 mg, still further optionally at least 100 mg, still further optionally at least 200 mg, still further optionally at least 400 mg, still further optionally at least 600 mg, still further optionally at least 800 mg still further optionally at least 1000 mg, still further optionally at least 1200 mg, still further optionally at least 1400 mg, still further optionally at least 1500 mg Omega-3 polyunsaturated fatty acid per 100 g of animal meat.

Optionally, the composition or method is for use in enriching animal meat with at least 40 mg Omega-3 polyunsaturated fatty acid per 100 kcal equivalent weight of animal meat. Further optionally, the composition or method is for use in enriching animal meat with 40-1500 mg Omega-3 polyunsaturated fatty acid per 100 kcal equivalent weight of animal meat. Still further optionally, the composition or method is for use in enriching animal meat with at least 50 mg, optionally at least 60 mg, further optionally at least 70 mg, still further optionally at least 80 mg, still further optionally at least 90 mg, still further optionally at least 100 mg, still further optionally at least 200 mg, still further optionally at least 400 mg, still further optionally at least 600 mg, still further optionally at least 800 mg still further optionally at least 1000 mg, still further optionally at least 1200 mg, still further optionally at least 1400 mg, still further optionally at least 1500 mg Omega-3 polyunsaturated fatty acid per 100 kcal equivalent weight of animal meat.

Optionally or additionally, the composition or method is for use in enriching animal meat with at least 40 mg docosahexaenoic acid (DHA) per 100 kcal equivalent weight of animal meat. Further optionally, 30 the composition or method is for use in enriching animal meat with 40-200 mg docosahexaenoic acid (DHA) per 100 kcal equivalent weight of animal meat. Still further optionally, the composition or method is for use in enriching animal meat with at least 50 mg, optionally at least 60 mg, further optionally at least 70 mg, still further optionally at least 80 mg, still further optionally at least 90 mg, still further optionally at least 100 mg, still further optionally at least 200 mg docosahexaenoic acid (DHA) per 100 kcal equivalent weight of animal meat.

Optionally or additionally, the composition or method is for use in enriching animal meat with at least 40 mg docosahexaenoic acid (DHA) per 100 g of animal meat. Further optionally, the composition or method is for use in enriching animal meat with 40-200 mg docosahexaenoic acid (DHA) per 100 g 40 of animal meat. Still further optionally, the composition or method is for use in enriching animal meat with at least 50 mg, optionally at least 60 mg, further optionally at least 70 mg, still further optionally at least 80 mg, still further optionally at least 90 mg, still further optionally at least 100 mg, still further optionally at least 200 mg docosahexaenoic acid (DHA) per 100 g of animal meat.

Optionally or additionally, the composition or method is for use in enriching animal meat with at least 40 mg eicosapentaenoic acid (EPA) per 100 kcal equivalent weight of animal meat. Further optionally, the composition or method is for use in enriching animal meat with 40-200 mg eicosapentaenoic acid (EPA) per 100 kcal equivalent weight of animal meat. Still further optionally, the composition or method is for use in enriching animal meat with at least 50 mg, optionally at least 60 mg, further optionally at least 70 mg, still further optionally at least 80 mg, still further optionally at least 90 mg, still 10 further optionally at least 100 mg, still further optionally at least 200 mg eicosapentaenoic acid (EPA) per 100 kcal equivalent weight of animal meat.

Optionally or additionally, the composition or method is for use in enriching animal meat with at least 40 mg eicosapentaenoic acid (EPA) per 100 g of animal meat. Further optionally, the composition or method is for use in enriching animal meat with 40-200 mg eicosapentaenoic acid (EPA) per 100 g of animal meat. Still further optionally, the composition or method is for use in enriching animal meat with at least 50 mg, optionally at least 60 mg, further optionally at least 70 mg, still further optionally at least 80 mg, still further optionally at least 90 mg, still further optionally at least 100 mg, still further optionally at least 200 mg eicosapentaenoic acid (EPA) per 100 g of animal meat.

Optionally or additionally, the composition or method is for use in enriching animal meat with at least 250 mg a-linolenic acid (ALA) per 100 g of animal meat. Further optionally, the composition or method is for use in enriching animal meat with 250-1500 mg α-linolenic acid (ALA) per 100 g of animal meat. Still further optionally, the composition or method is for use in enriching animal meat with at least 300 mg, optionally at least 400 mg, further optionally at least 600 mg, still further optionally at least 800 mg still further optionally at least 1000 mg, still further optionally at least 1200 mg, still further optionally at least 1400 mg, still further optionally at least 1500 mg α-linolenic acid (ALA) per 100 g of animal meat.

Optionally or additionally, the composition or method is for use in enriching animal meat with at least 250 mg α-linolenic acid (ALA) per 100 kcal equivalent weight of animal meat. Further optionally, the composition or method is for use in enriching animal meat with 250-1500 mg α-linolenic acid (ALA) per 100 kcal equivalent weight of animal meat. Still further optionally, the composition or method is for use in enriching animal meat with at least 300 mg, optionally at least 400 mg, further optionally at least 600 mg, still further optionally at least 800 mg still further optionally at least 1000 mg, still further optionally at least 1200 mg, still further optionally at least 1400 mg, still further optionally at least 1500 mg α-linolenic acid (ALA) per 100 kcal equivalent weight of animal meat.

According to a further aspect of the present invention there is provided a method for enriching animal meat with Omega-3 polyunsaturated fatty acids, the method comprising the steps of administering a composition according to a first aspect of the invention or an animal feed according to a second aspect of the invention to an animal.

According to a fourth aspect of the present invention there is provided animal meat enriched with Omega-3 polyunsaturated fatty acids for increasing the Omega-3 polyunsaturated fatty acids levels in human plasma after consumption of the enriched poultry meat and subsequent reductions in the risk factors for cardiovascular disease.

According to a fifth aspect of the present invention there is provided a method of increasing the Omega-3 polyunsaturated fatty acids levels in human plasma, the method comprising administering animal meat enriched with Omega-3 polyunsaturated fatty acids to a subject.

Optionally, the method comprises oral administration of the animal meat enriched with Omega-3 polyunsaturated fatty acids to the subject.

Optionally, the method comprises dietary administration of the animal meat enriched with Omega-3 polyunsaturated fatty acids to the subject.

Optionally, the animal meat is enriched with Omega-3 polyunsaturated fatty acids using a composition according to a first aspect of the present invention or an animal feed according to a second aspect of the present invention; or by a method according to a third or fifth aspect of the invention.

Optionally, the animal meat enriched with Omega-3 polyunsaturated fatty acids is prepared according to the method according to the fourth aspect of the present invention.

### EXAMPLES

Embodiments of the present invention will now be described with reference to the following non-limiting examples.

### Example 1

### Study 1. Poultry-Meat Enrichment With DHA and EPA

The trial was carried out to assess the level of enrichment of EPA and DHA in poultry meat after using various sources and levels of EPA and DHA in the poultry diet. The eating quality of the broiler meat was also examined.

972 Ross 308 birds, split housed and sexed were used in this trial. The day old chicks were delivered from commercial hatcheries. They were fed ad libitum with tube feeders with hoppers and nipple drinkers. All birds had 23 hours of light per day from day 0 to day 7, and 18 hours of light for the remainder of the crop. The birds were vaccinated at day 18.

Three groups of poultry were fed one of 3 diets:
Control diet (T1)
Control diet with 7.5% (w/w) composition of the invention (T2)
Control diet with 15.0% (w/w) composition of the invention (T3)

TABLE 1
Formulation of the control Diet (T1)

| | | | Broiler |
|---|---|---|---|
| | Broiler | Broiler | Finisher/ |
| | Starter | Grower | Withdrawal |
| Maize | 15.0 | 10.0 | 10.0 |
| Wheat | 47.9 | 55.8 | 59.4 |
| Full fat soya | 12.5 | 12.5 | 12.5 |
| Soya bean meal | 19.1 | 15.3 | 11.5 |
| Soyabean oil | 1.35 | 2.64 | 2.89 |
| Calcium carbonate, dicalcium | 2.900 | 2.800 | 2.700 |
| phosphate, sodium bicarbonate, | | | |
| sodium chloride, vitamins and minerals | | | |
| Amino acids | 1.100 | 1.000 | 1.000 |

The composition of the invention comprised % (w/w):

| Group description | % in OMP |
|---|---|
| Linseed micronised | 66.7 |
| Dehydrated algae cells | 8.0 |
| Protected fish oil | 3.3 |
| Surfactant/emulsifier/binder/flow agent | 5.3 |
| Synthetic/Natural Antioxidants | 2.0 |
| Cereal base | 14.7 |

l The composition of the invention was added directly into the feed during production at 7.5% and 15% to give experimental diets T2 and T3, respectively.

Fatty acid analysis of the meat portions was carried out using gas chromatography via methyl esters. The method for fatty acid hydrolysis is British Standard 4401 Pt 4:1970. Tests are UKAS accredited to BS ENO/IE17025:2005.The samples were analysed in Mylnefield Research Services Ltd, Dundee, Scotland. Mylnefield Research Services Ltd, is an accredited laboratory under ISO:17025.

Sensory analysis was carried out by Wirral Sensory Services Ltd. A Central Location Test of 100 typical consumers was carried out (regular consumers of whole chickens with a mix of age, gender and socio-economic demographics). The respondents were presented with the products in a sequential monadic order; the products were de-branded before being given to the respondents and the order of presentation was rotated to prevent any potential bias. Respondents were then asked to score each of the products for a number of key parameters on a 0-10 point hedonic scale, as well as noting down any specific likes and dislikes. They were also asked to score the products on a 5-point diagnostic scale for certain parameters to offer a greater understanding. Results of the fatty acid analysis are shown in Table 2, and results of the sensory analysis are shown in Table 3.

TABLE 2
Average sum of DHA + EPA (mg)/100
g meat in broiler meat portions

| | Breast | | Thigh | Drumstick | Wings |
|---|---|---|---|---|---|
| | +skin | −skin | +skin | +skin | +skin |
| T1 (Control) | 41.3 | 30.2 | 56.3 | 47.1 | 57.5 |
| T2 (+7.5% fat premix) | 83.5 | 57.2 | 135.6 | 104.3 | 162.7 |
| T3 (+15% fat premix) | 91.3 | 55.0 | 151.4 | 79 | 148.7 |

Table 3 sensory results (10-point acceptance scale; results show mean score where 1=Extremely Unacceptable, 10=Extremely Acceptable)

| | Control (T1) | T2 | T3 |
|---|---|---|---|
| Overall | 7.6^{a} | 8.02^{b} | 7.34^{b} |
| Appearance | 7.4^{a} | 7.87^{b} | 7.72^{ab} |
| Aroma | 7.09 | 7.44 | 7.39 |
| Texture | 7.39 | 7.79 | 7.45 |
| Moisture | 7.44^{a} | 8.14^{b} | 6.94^{c} |
| Succulence | 7.5^{a} | 7.88^{a} | 6.87^{b} |
| Tenderness | 7.91^{ab} | 7.57^{a} | 8.23^{b} |
| Flavour | 7.39 | 7.77 | 7.45 |
| Preference (%) | 31 | 44 | 18 |

^{a,b,c}values are significantly different at P < 0.05, means that share the same superscript are significantly different from each other

Supplementation with high levels of omega fatty acids (up to 15%% of the composition of the invention in the diet) resulted in enrichment of the meat. Analysis of the cooked meat showed no significant differences in texture or in flavour between the 3 treatments. However, the enriched meat from the supplementation at 7.5% composition of the invention was overall the most acceptable product, showing differences between the treatments in succulence, moistness, tenderness and visual score (see Table 3). This meat was also the most preferred meat.

### Example 2

### Optimisation of Feed, Bird Performance and Human Health Benefits

A trial was carried out as a feed production study, a study on bird performance and a clinical human study. The aims were to optimise the formulation of the composition of the invention, to assess the effects of dietary supplementation with the composition of the invention on bird production performance, to study the time course of absorption and accumulation of chicken-meat derived omega-3 PUFAs in humans and to look at the effects of omega-enriched chicken meat on clinical measurements of the reduction of the risk factors of cardiovascular health. The composition of the invention was optimised by increasing the level of fish oil and reducing the level of linseed (Table 5).

22,800 Ross birds, split housed and sexed were used in the trial. The day old chicks were delivered from commercial hatcheries. They were fed and watered ad libitum with automatic feeders and nipple drinkers. All birds had 23 hours of light per day from day 0 to day 7, and 18 hours of light for the remainder of the crop. The birds were vaccinated at day 18. Birds were fed a standard starter and grower diet (same as control starter and grower diet) and then offered a finisher ration containing 10% (w/w) of a composition of the invention as defined in Table 4 from 21 days for approximately 20 days (until final kill).

TABLE 4

| | | | Broiler |
|---|---|---|---|
| | Broiler | Broiler | Finisher/ |
| Control diet | Starter | Grower | Withdrawal |
| Maize | 15.0 | 10.0 | 10.0 |
| Wheat | 47.9 | 55.8 | 59.4 |
| Full fat soya | 12.5 | 12.5 | 12.5 |
| Soya bean meal | 19.1 | 15.3 | 11.5 |
| Soyabean oil | 1.35 | 2.64 | 2.89 |
| Calcium carbonate, dicalcium | 2.900 | 2.800 | 2.700 |
| phosphate, sodium bicarbonate, | | | |
| sodium chloride, vitamins and minerals | | | |
| Amino acids | 1.100 | 1.000 | 1.000 |

TABLE 5
Adapted omega premix formulation

| — | % in OMP |
|---|---|
| Linseed micronised | 30.0 |
| Dehydrated algae cells | 7.5 |
| Protected fish oil | 20.0 |
| Surfactant/emulsifier/binder/flow agent | 4.0 |
| Synthetic/Natural Antioxidants | 1.5 |
| Cereal base | 37.0 |

Male birds were selected for the trial. Birds were processed, this typically involves stunning, bleeding, spay washing, de-feathering, scalding, head/foot removal, evisceration, carcass inspection, spray washing, primary chilling, weighing and secondary chilling. The birds were then portioned as required and frozen until required by the study participants or minced and sent to the laboratory for fatty acid profiling.

Thirty healthy participants were recruited to this open sequential study. Blood and urine sampling, blood pressure and heart rate measurements, occurred at baseline, and after 1, 3 and 5 weeks of eating 3 servings/week of omega-3 enriched chicken-meat. Participants received enriched chicken sufficient for 3 meals a week for 5 weeks. Each week each participant family received 1 whole chicken, 4 breasts, 4 thighs, 4 drumsticks, 4 wings plus skin. Study participants (adults 18 years) were selected according to the following exclusion criteria:1) Previous cardiovascular disease (myocardial infarction, coronary intervention, or stroke), 2) Currently prescribed anti-hypertensive, lipid lowering, or anti-platelet therapy and, 3) Currently regularly taking Omega-3 polyunsaturated fatty acid supplements.

Blood samples were sent to Mylnefield Lipid Analysis an external laboratory (Accredited to IS09001:2008). Plasma and red cell levels of C10:0 Capric acid, C10:1(n-1)cis cis-9-Decenoic acid, C12:0 Lauric acid, C12:1(n-1)cis cis-11-Dodecenoic acid, C12:1(n-3)cis cis-9-Dodecenoic acid, C13:0 Tridecanoic acid, C14:0 ante-iso 11-Methyltridecanoic acid, C14:0 iso 12-Methyltridecanoic acid, C14:0 Myristic acid, C14:1(n-5)cis Myristoleic Acid, C15:0 ante-iso 12-Methyltetradecanoic acid, C15:0 iso 13-Methylmyristic acid, C15:0 Pentadecanoic acid, C15:1(n-5)cis cis-10-Pentadecenoic Acid, C16:0 iso 14-Methylpentadecanoic acid, C16:0 Palmitic acid, C16:1(n-5)cis cis-11-Hexadecenoic acid, C16:1(n-7)cis Palmitoleic acid C16:1(n-9)cis cis-5-Hexadecenoic acid, C17:0 ante-iso 14-Methylhexadecanoic acid, C17:0 Heptadecanoic acid, C17:0 iso 15-Methylpalmitic acid, C17:1(n-7)cis cis-10-Heptadecenoic Acid, C18:0 ante-iso 15-Methylheptadecanoic acid, C18:0 iso 16-Methylheptadecanoic acid, C18:0 Stearic acid, C18:1(n-11)trans trans-7-Octadecenoic acid, C18:1(n-6)cis cis-12-Octadecenoic acid, C18:1(n-6)trans trans-12-Octadecenoic Acid, C18:1(n-7)cis cis-Vaccenic Acid, C18:1(n-7)trans trans-Vaccenic acid, C18:1(n-9)cis Oleic acid, C18:1(n-9)trans Elaidic acid, C18:2(n-6)cis Linoleic acid, C18:2(n-6)trans Linolelaidic acid, C18:2conj Total Conjugated Linoleic acid (CLA), C18:3(n-3)cis Alpha-Linolenic acid (ALA), C18:3(n-6)cis Gamma-Linolenic acid (GLA), C18:4(n-3)cis Stearidonic acid, C20:0 Arachidic acid, C20:1(n-11) Gadoleic Acid, C20:1(n9)cis cis-11-Eicosenoic Acid, C20:2(n-6)cis cis-11,14-Ecosadienoic acid, C20:3(n-3)cis cis-11,14,17-Eicosatrienoic acid, C20:3(n-6)cis cis-8,11,14 Eicosatrienoic acid, C20:4(n-3)cis cis-8,11,14,17-Eicosatetraenoic acid, C20:4(n-6)cis Arachidonic Acid, C20:5(n-3)cis Eicosapentenoic acid (EPA), C22:0 Behenic acid, C22:1(n-11)cis Cetoleic acid, C22:1(n-9)cis Erucic acid, C22:2(n-6)cis Docosadienoic acid, C22:4(n, 6)cis Docosatetraenoic acid, C22:5(n-3)cis Docosapentaenoic (DPA), C22:5(n-6)cis cis4,7,10,13,16Docosapentaenoic acid, C22:6(n-3)cis Docosahexaenoic (DHA), C24:0 Lignoceric acid, C24:1(n-9)cis Nervonic acid, C25:0 Pentacosanoic acid, C4:0 Butyric acid, C5:0 Valeric acid, C6:0 Caproic acid, C7:0 Heptanoic acid, C8:0 Caprylic acid, C9:0 Nonanoic acid were measured.

Additionally, the following Cardiovascular Risk Factors were recorded at Beaumont Hospital, Dublin at the start and at the end (week 5) of the trial: weight and waist circumference, office blood pressure heart rate, plasma lipids (triglyceride levels), plasma C-reactive protein, platelet function 40 and turnover (baseline and week 5): urinary thromboxane, light transmission aggregometry, ADP secretion assay, serum thromboxane. All measurements were taken according to Beaumont Hospital standard operating procedures.

Poultry performance results showed that birds receiving the a composition of the invention showed similar growth rates, feed efficiency and mortality as compared to birds receiving standard commercial diets:

TABLE 6
Performance results, whole house

| | Omega-premix | |
|---|---|---|
| Bodyweight | 2.2 | kg |
| FCR | 1.63 | |
| Ave age at slaughter | 37.48 | days |

| | |
|---|---|
| Mortality | 1.47% |
| EFEP | 355 |

TABLE 7
Average sum of DHA + EPA (mg)/100
g meat in broiler meat portions

average sum DHA +
EPA (mg)/100 g

| Portion | Control | Omega Premix |
|---|---|---|
| Whole bird | 14.40 | 77.85 |
| Deboned fillet + skin | 12.95 | 70.43 |
| Deboned fillet − skin | 10.28 | 55.8 |
| Deboned thigh + skin | 17.23 | 116.45 |
| Deboned thigh − skin | 14.65 | 122.08 |
| Drums + skin | 11.25 | 82.43 |
| Wings + skin | 11.45 | 83.8 |

High levels of enrichment in all of the meat portions were achieved.

Results of the clinical study are shown in table 8:

| | Baseline | 5 Weeks | Change | p-value |
|---|---|---|---|---|
| Weight (kg) | 71.6 ± 10.5 | 71.5 ± 10.9 | −0.5 | 0.848 |
| BMI (kg/m²) | 24.6 ± 2.9 | 24.6 ± 3.2 | — | 0.878 |
| Waist (cm) | 84.1 ± 8.8 | 85.1 ± 8.7 | +1.0 | 0.283 |
| Clinic BP | | | | |
| Systolic (mm Hg) | 116.1 ± 11.6 | 113.5 ± 10.9 | −2.6 | 0.014* |
| Diastolic (mm Hg) | 68.9 ± 7.7 | 69.1 ± 7.5 | +0.2 | 0.862 |
| Pulse pressure | 47.2 ± 8.9 | 44.4 ± 9.2 | −2.8 | 0.002** |
| (mm Hg) | | | | |
| Heart Rate (beats/min) | 65.7 ± 7.6 | 64.3 ± 9.4 | −1.4 | 0.361 |
| C-Reactive Protein | 1.2 ± 1.0 | 1.6 ± 1.5 | +0.4 | 0.182 |
| (mg/L) | | | | |
| Urinary Thromboxane | 1435 ± 1384 | 1024 ± 800 | −411 | 0.037* |
| (pg/mg creatinine) | | | | |

Significantly different
*P < 0.05,
**P < 0.05

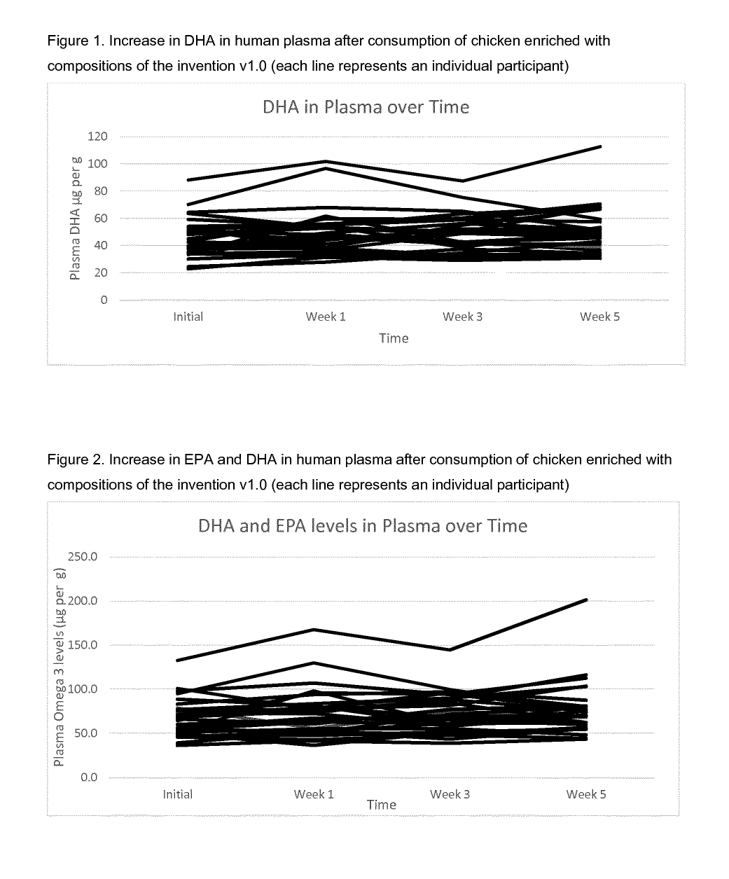

In terms of the bio-markers for health, the mean systolic BP reduced from 116 to 113 mmHg (p=0.014), and the mean urinary thromboxane/creatinine ratio (measure of platelet turnover) reduced from 1435 to 1024 pg/mg (p=0.037). The DHA and EPA levels increased from 82 mg/g at baseline to 93 mg/g at five weeks (12% increment, p=0.006). See FIGS. 1 and 2

### Example 3

### Optimisation For Maximum Enrichment—Replacement of Fish Oil and Sensory Analysis

A trial was carried out to further modify the dietary composition of the invention in order to achieve the maximum enrichment of all meat portions including the breast meat. The diets were further modified to try to achieve enrichment of the poultry meat without the use of fish oil, in order to meet the needs of birds for which animal-derived ingredients are not permitted.

2268 Ross 308 split housed and sexed were used in the trial. The day old chicks were delivered from commercial hatcheries. They were fed ad libitum with tube feeders with hoppers and nipple drinkers. All birds had 23 hours of light per day from day 0 to day 7, and 18 hours of light for the remainder of the crop. The birds were vaccinated at day 18.The birds were, divided into 7 treatments. The treatments were

T1. Control, standard diet

T2. Omega Premix A fed during finisher and withdrawal periods

T3. Omega Premix B fed during finisher and withdrawal periods

T4. Omega Premix C fed during finisher and withdrawal periods

T5. Omega Premix D fed during finisher and withdrawal periods

T6. Omega Premix E fed during finisher and withdrawal periods

T7. Omega Premix A fed during grower, finisher and withdrawal periods

TABLE 9

| | | | Broiler |
|---|---|---|---|
| | Broiler | Broiler | Finisher/ |
| | Starter | Grower | withdrawal |
| Maize | 15.0 | 10.0 | 10.0 |
| Wheat | 47.9 | 55.8 | 59.4 |
| Full fat soya | 12.5 | 12.5 | 12.5 |
| Soya bean meal | 19.1 | 15.3 | 11.5 |
| Soyabean oil | 1.35 | 2.64 | 2.89 |
| Calcium carbonate, dicalcium | 2.900 | 2.800 | 2.700 |
| phosphate, sodium bicarbonate, | | | |
| sodium chloride, vitamins and minerals | | | |
| Amino acids | 1.100 | 1.000 | 1.000 |

The compositions of the invention used are shown in Table 10. Compositions of the invention were added into the final feed at 10% of the total diet. Bird performance was recorded by the trial investigator daily and at slaughter.

30 composite samples were sent for meat analysis. This involved stunning, bleeding, spay washing, de-feathering, scalding, head/foot removal, evisceration, carcass inspection, spray washing, primary chilling, weighing and secondary chilling. The birds were then portioned as required and sent to Agri-Food and BioSciences Institute (AFBI), Belfast, Northern Ireland for fatty acid profiling. Fatty acid analysis of the meat portions was carried out using gas chromatography via methyl esters. The fatty acids measured were: C10:0 Capric acid, C10:1(n-1)cis cis-9-Decenoic acid, C12:0 Lauric acid, C12:1(n-1)cis cis-11-Dodecenoic acid, C12:1(n-3)cis cis-9-Dodecenoic acid, C13:0 Tridecanoic acid, C14:0 ante-iso 11-Methyltridecanoic acid, C14:0 iso 12-Methyltridecanoic acid, C14:0 Myristic acid, C14:1(n-5)cis Myristoleic Acid, C15:0 ante-iso 12-Methyltetradecanoic acid, C15:0 iso 13-Methylmyristic acid, C15:0 Pentadecanoic acid, C15:1(n-5)cis cis-10-Pentadecenoic Acid, C16:0 iso 14-Methylpentadecanoic acid, C16:0 Palmitic acid, C16:1(n-5)cis cis-11-Hexadecenoic acid, C16:1(n-7)cis Palmitoleic acid, C16:1(n-9)cis cis-5-Hexadecenoic acid, C17:0 ante-iso 14-Methylhexadecanoic acid, C17:0 Heptadecanoic acid, C17:0 iso 15-Methylpalmitic acid, C17:1(n-7)cis cis-10-Heptadecenoic Acid, C18:0 ante-iso 15-Methylheptadecanoic acid, C18:0 iso 16-Methylheptadecanoic acid, C18:0 Stearic acid, C18:1(n-11)trans trans-7-Octadecenoic acid, C18:1(n-6)cis cis-12-Octadecenoic acid, C18:1(n-6)trans trans-12-Octadecenoic Acid, C18:1(n-7)cis cis-Vaccenic Acid, C18:1(n-7)trans trans-Vaccenic acid, C18:1(n-9)cis Oleic acid, C18:1(n-9)trans Elaidic acid, C18:2(n-6)cis Linoleic acid, C18:2(n-6)trans Linolelaidic acid, C18:2conj Total Conjugated Linoleic acid (CLA), C18:3(n-3)cis Alpha-Linolenic acid (ALA), C18:3(n-6)cis Gamma-Linolenic acid (GLA), C18:4(n-3)cis Stearidonic acid, C20:0 Arachidic acid, C20:1(n-11) Gadoleic Acid, C20:1(n9)cis cis-11-Eicosenoic Acid, C20:2(n-6)cis cis-11,14-Ecosadienoic acid, C20:3(n-3)cis cis-11,14,17-Eicosatrienoic acid, C20:3(n-6)cis cis-8,11,14 Eicosatrienoic acid, C20:4(n-3)cis cis-8,11,14,17-Eicosatetraenoic acid, C20:4(n-6)cis Arachidonic Acid, C20:5(n-3)cis Eicosapentenoic acid (EPA), C22:0 Behenic acid, C22:1(n-11)cis Cetoleic acid, C22:1(n-9)cis Erucic acid, C22:2(n-6)cis Docosadienoic acid, C22:4(n, 6)cis Docosatetraenoic acid, C22:5(n-3)cis Docosapentaenoic (DPA), C22:5(n-6)cis cis4,7,10,13,16Docosapentaenoic acid, C22:6(n-3)cis Docosahexaenoic (DHA), C24:0 Lignoceric acid, C24:1(n-9)cis Nervonic acid, C25:0 Pentacosanoic acid, C4:0 Butyric acid, C5:0 Valeric acid, C6:0 Caproic acid, C7:0 Heptanoic acid, C8:0 Caprylic acid, C9:0 Nonanoic acid. The method for fatty acid hydrolysis is British Standard 4401 Pt 4:1970. The tests are UKAS accredited to BS ENO/IE17025:2005.The samples were analysed in Eurofins Scientific, an accredited laboratory under ISO:17025.

In addition, sensory analysis was carried out using (blind-coded) chicken. The chicken was cooked in the oven at 190° C. until a minimum deep thigh muscle temperature of 86° C. was achieved. Sensory analysis were carried out by Wirral Sensory Services Ltd. A Central Location Test of 109 typical consumers was carried out (regular consumers of whole chickens with a mix of age, gender and socio-economic demographics). The respondents were presented with the products in a sequential monadic order; the products were de-branded before being given to the respondents and the order of presentation was rotated to prevent any potential bias. Respondents were then asked to score each of the products for a number of key parameters on a 0-10 point hedonic scale, as well as noting down any specific likes and dislikes. They were also asked to score the products on a 5-point diagnostic scale for certain parameters to offer a greater understanding.

TABLE 10
Composition Formulations

| | A | B | C | D | E |
|---|---|---|---|---|---|
| Micronised linseed | 30 | 20 | 30 | 15 | 30 |
| Dehydrated algae | 7.5 | 7.5 | 15 | 15 | 15 |
| Encapsulated fish oil | 20 | 40 | 20 | 40 | 0 |
| Surfactant/emulsifier/ | 4 | 4 | 4 | 4 | 4 |
| binder/flow agent | | | | | |
| Synthetic/Natural | 1.5 | 1.5 | 1.5 | 1.5 | 1.5 |
| Antioxidants | | | | | |
| Cereal | 37 | 27 | 29.5 | 24.5 | 49.5 |

Results of bird performance are shown in tables 11 and 12.

TABLE 11
Live weight gain (g/bird)

| | Control | | | | | | |
|---|---|---|---|---|---|---|---|
| | T1 | T2 | T3 | T4 | T5 | T6 | T7 |
| 7 days | 180 | 189 | 183 | 183 | 186 | 181 | 191 |
| 14 days | 480 | 507 | 499 | 519 | 499 | 499 | 512 |
| 21 days | 935 | 974 | 924 | 945 | 958 | 967 | 997 |
| 28 days | 1478 | 1561 | 1470 | 1563 | 1476 | 1549 | 1606 |
| Av 36.5 d | 2113 | 2083 | 2068 | 2089 | 2061 | 2012 | 2059 |
| | (37 d) | | | | | | |

TABLE 12
Feed conversion ratio

| | Control | | | | | | |
|---|---|---|---|---|---|---|---|
| | T1 | T2 | T3 | T4 | T5 | T6 | T7 |
| 7 days | 0.828 | 0.834 | 0.826 | 0.834 | 0.833 | 0.855 | 0.865 |
| 14 days | 1.142 | 1.118 | 1.081 | 1.053 | 1.112 | 1.086 | 1.102 |
| 21 days | 1.282 | 1.287 | 1.285 | 1.326 | 1.272 | 1.240 | 1.212 |
| 28 days | 1.397 | 1.423 | 1.444 | 1.438 | 1.493 | 1.411 | 1.391 |
| Av 36.5 d | 1.571 | 1.613 | 1.608 | 1.606 | 1.607 | 1.644 | 1.638 |
| | (37 d) | | | | | | |

Fat analysis of the chicken meat from two different laboratories is shown in table 13, while taste panel results are shown in tables 14 and 15:

TABLE 13
Sum of DHA + EPA (mg)/100 g meat in broiler meat portions

| | Breast | | Thigh | |
|---|---|---|---|---|
| | +skin | −skin | +skin | −skin |
| T1 | 12.95 | 10.28 | 17.23 | 14.65 |
| T2 | 52.72 | 40.25 | 60.46 | 59.28 |
| T3 | 65.81 | 59.97 | 94.41 | 104.99 |
| T4 | 86.17 | 79.7 | 78.49 | 130.69 |
| T5 | 110.84 | 95.32 | 217.62 | 176.94 |
| T6 | 94.23 | 76.55 | 121.35 | 95.03 |
| T7 | 61.43 | 52.27 | 85.16 | 52.27 |

TABLE 14
Taste panel results; mean scores for product
attributes of white meat samples

| | T1 | | | | | | |
|---|---|---|---|---|---|---|---|
| Attribute | Control | T2 | T3 | T4 | T5 | T6 | T7 |
| Cooked | 6.89a | 7.21a | 7.16a | 7.37a | 7.21a | 7.21a | 7.00a |
| appearance | | | | | | | |
| Aroma | 6.89a | 7.21a | 6.68a | 7.16a | 7.21a | 7.00a | 6.74a |
| Taste | 6.58a | 7.21a | 6.68a | 7.26a | 7.11a | 7.32a | 6.95a |
| After taste | 6.37a | 6.68a | 6.47a | 6.95a | 7.05a | 7.16a | 6.58a |
| Texture | 5.74b | 6.74a | 6.32ab | 6.95a | 6.89a | 6.95a | 6.58ab |
| Succulence | 5.74a | 6.00a | 6.00a | 6.53a | 6.74a | 6.63a | 6.16a |
| Overall | 6.00b | 6.68ab | 6.26ab | 6.89a | 6.89a | 7.11a | 6.68ab |
| accept- | | | | | | | |
| ability | | | | | | | |

TABLE 15
Taste panel results; mean scores for product
attributes of dark meat samples

| | T1 | | | | | | |
|---|---|---|---|---|---|---|---|
| Attribute | Control | T2 | T3 | T4 | T5 | T6 | T7 |
| Cooked | 7.07a | 7.14a | 7.21a | 7.00a | 7.00a | 7.00a | 7.14a |
| appearance | | | | | | | |
| Aroma | 7.00a | 7.07a | 6.57a | 6.93a | 7.07a | 7.07a | 7.21a |
| Taste | 7.14a | 7.14a | 6.71ab | 7.21a | 5.71b | 6.29ab | 7.00ab |
| After taste | 6.79ab | 6.86ab | 6.36ab | 6.93a | 5.50b | 6.14ab | 6.86ab |
| Texture | 6.86a | 7.21a | 6.57a | 6.36a | 6.36a | 6.29a | 6.93a |
| Succulence | 6.64a | 7.07a | 6.36a | 6.29a | 6.14a | 6.50a | 6.93a |
| Overall | 6.86a | 7.07a | 6.21ab | 6.43ab | 5.43b | 6.00ab | 6.71ab |
| accept- | | | | | | | |
| ability | | | | | | | |

There were no significant differences between treatments for the white or dark meat samples with regards to the cooked appearance, aroma, or succulence. There were slight significant differences noted with regards to texture and overall acceptability of white meat, with the high algal oil-fed birds, and the zero fish oil-fed birds scoring better than the control birds for both of these attributes. There were some slight significant differences noted with regards to taste, after taste and overall acceptability of dark meat; birds receiving the high fish oil, high algal—oil treatment scored worse for taste, aftertaste and overall acceptability.

### Example 4

### Refining the Composition of the Invention and to Evaluate Alternative Sources of Omega 3 Sources

A trial was carried out to refine the composition of the invention and to evaluate alternative sources of Omega 3 sources (micronized linseed, dehydrated algae and algal oil). 972 Ross 308 birds sexed and placed in 3 pens with 324 birds per pen. The day old chicks were delivered from commercial hatcheries. They were fed ad libitum with tube feeders with hoppers and nipple drinkers. All birds had 23 hours of light per day from day 0 to day 7, and 18 hours of light for the remainder of the crop. The birds were vaccinated at day 18. They were fed a standard commercial starter and grower diet. Omega-enriched diets were fed from day 22, at 10% of the total diet. Birds were thinned at 35 days and final slaughter was at 39 days.

Treatments were:
Control
T1 : 10% Premix 1 from day 22 to end (Finisher and withdrawal)
T2 10% Premix 2 from day 22 to end (Finisher and withdrawal)

Formulae of the premixes is shown in table 21.

TABLE 21
Premix formulations

| | Premix 1 | Premix 2 |
|---|---|---|
| Micronized linseed | 30 | 15 |
| Algal oil | 6.9 | 5.2 |
| Surfactant, emulsifier, binder and flow agent | 1 | 1 |
| Synthetic and natural antioxidants | 1.5 | 1.5 |
| Cereals | 59 | 75.8 |
| Inclusion rate in feed % | 10 | 10 |
| C18:2% in feed | 0.58 | 0.30 |
| C20:5 + C22:6% in feed | 0.27 | 0.20 |

Results: Performance results are shown in table 22 and 23.

TABLE 22
Bodyweight gain g/day

| | Control Av (g/d) | T1(g/d) | T2 (g/d) |
|---|---|---|---|
| 7 d | 17.8 | 19.4 | 19.3 |
| 14 d | 41.7 | 45.7 | 43.6 |
| 21 d | 67.1 | 66.8 | 72.8 |
| 28 d | 83.0 | 91.3 | 68.0 |
| 35 d | 74.0 | 73.3 | 86.4 |
| 37 d Av | 88.8 | 63.8 | 80.8 |

TABLE 23
Feed Conversion Ratio

| | Control Av | T1 | T2 |
|---|---|---|---|
| 7 d | 0.88 | 0.87 | 0.91 |
| 14 d | 1.16 | 1.10 | 1.17 |
| 21 d | 1.13 | 1.26 | 1.26 |
| 28 d | 1.45 | 1.35 | 1.46 |
| 35 d | 1.65 | 1.53 | 1.56 |
| 37 d Av | 1.66 | 1.61 | 1.59 |

TABLE 24
Omega 3 deposition in the meat for treatment 1 and 2

| | |
|---|---|
| Average | Average |
| Treatment 1 | Treatment 2 |

BREAST

| | | |
|---|---|---|
| EPA mg/100 g | 9.7 | 9.2 |
| DHA mg/100 g | 70.6 | 65.7 |

THIGH

| | | |
|---|---|---|
| EPA mg/100 g | 36.3 | 30.4 |
| DHA mg/100 g | 202.4 | 173.6 |

The algae oil and linseed are capable of enriching chicken meat.

### Example 5

### Fish-Free Compositions

A trial was carried out to compare results from an Omega 3 enrichment of chicken with and without fish oil or algal oil.

972 Ross 308 split housed and sexed were used in the trial. The day old chicks were delivered from commercial hatcheries. They were fed ad libitum with tube feeders with hoppers and nipple drinkers. All birds had 23 hours of light per day from day 0 to day 7, and 18 hours of light for the remainder of the crop. The birds were vaccinated at day 18.The birds were, divided into 3 treatments. The treatments were
T1. Control, standard diet
T2. Omega Premix A fed during finisher and withdrawal periods
T3. Omega Premix B fed during finisher and withdrawal periods

Compositions according to the present invention were prepared as indicated in Tables 26 below.

| | | | Broiler |
|---|---|---|---|
| | Broiler | Broiler | Finisher/ |
| T25. Control diet | Starter | Grower | withdrawal |
| Maize | 15.0 | 10.0 | 10.0 |
| Wheat | 47.9 | 55.8 | 59.4 |
| Full fat soya | 12.5 | 12.5 | 12.5 |
| Soya bean meal | 19.1 | 15.3 | 11.5 |
| Soyabean oil | 1.35 | 2.64 | 2.89 |
| Calcium carbonate, dicalcium | 2.900 | 2.800 | 2.700 |
| phosphate, sodium bicarbonate, | | | |
| sodium chloride, vitamins and minerals | | | |
| Amino acids | 1.100 | 1.000 | 1.000 |

TABLE 26
Composition of premixtures with and without fish oil

| Ingredient | Premix A (%) | Premix B (%) |
|---|---|---|
| Cereal | 37.0 | 49.5 |
| Micronised linseed | 30.0 | 30.0 |
| Fish oil (45%) | 20.0 | 0.0 |
| Microalgae | 7.5 | 15.0 |
| Anti-caking agent and antioxidants | 5.5 | 5.5 |

Compositions of the invention were added into the final feed at 10% of the total diet. Bird performance was recorded by the trial investigator daily and at slaughter.

30 composite samples were sent for meat analysis. This involved stunning, bleeding, spay washing, de-feathering, scalding, head/foot removal, evisceration, carcass inspection, spray washing, primary chilling, weighing and secondary chilling. The birds were then portioned as required and sent to Agri-Food and BioSciences Institute (AFBI), Belfast, Northern Ireland for fatty acid profiling. Fatty acid analysis of the meat portions was carried out using gas chromatography via methyl esters. The fatty acids measured were: C10:0 Capric acid, C10:1(n-1)cis cis-9-Decenoic acid, C12:0 Lauric acid, C12:1(n-1)cis cis-11-Dodecenoic acid, C12:1(n-3)cis cis-9-Dodecenoic acid, C13:0 Tridecanoic acid, C14:0 ante-iso 11-Methyltridecanoic acid, C14:0 iso 12-Methyltridecanoic acid, C14:0 Myristic acid, C14:1(n-5)cis Myristoleic Acid, C15:0 ante-iso 12-Methyltetradecanoic acid, C15:0 iso 13-Methylmyristic acid, C15:0 Pentadecanoic acid, C15:1(n-5)cis cis-10-Pentadecenoic Acid, C16:0 iso 14-Methylpentadecanoic acid, C16:0 Palmitic acid, C16:1(n-5)cis cis-11-Hexadecenoic acid, C16:1(n-7)cis Palmitoleic acid, C16:1(n-9)cis cis-5-Hexadecenoic acid, C17:0 ante-iso 14-Methylhexadecanoic acid, C17:0 Heptadecanoic acid, C17:0 iso 15-Methylpalmitic acid, C17:1(n-7)cis cis-10-Heptadecenoic Acid, C18:0 ante-iso 15-Methylheptadecanoic acid, C18:0 iso 16-Methylheptadecanoic acid, C18:0 Stearic acid, C18:1(n-11)trans trans-7-Octadecenoic acid, C18:1(n-6)cis cis-12-Octadecenoic acid, C18:1(n-6)trans trans-12-Octadecenoic Acid, C18:1(n-7)cis cis-Vaccenic Acid, C18:1(n-7)trans trans-Vaccenic acid, C18:1(n-9)cis Oleic acid, C18:1(n-9)trans Elaidic acid, C18:2(n-6)cis Linoleic acid, C18:2(n-6)trans Linolelaidic acid, C18:2conj Total Conjugated Linoleic acid (CLA), C18:3(n-3)cis Alpha-Linolenic acid (ALA), C18:3(n-6)cis Gamma-Linolenic acid (GLA), C18:4(n-3)cis Stearidonic acid, C20:0 Arachidic acid, C20:1(n-11) Gadoleic Acid, C20:1(n9)cis cis-11-Eicosenoic Acid, C20:2(n-6)cis cis-11,14-Ecosadienoic acid, C20:3(n-3)cis cis-11,14,17-Eicosatrienoic acid, C20:3(n-6)cis cis-8,11,14 Eicosatrienoic acid, C20:4(n-3)cis cis-8,11,14,17-Eicosatetraenoic acid, C20:4(n-6)cis Arachidonic Acid, C20:5(n-3)cis Eicosapentenoic acid (EPA), C22:0 Behenic acid, C22:1(n-11)cis Cetoleic acid, C22:1(n-9)cis Erucic acid, C22:2(n-6)cis Docosadienoic acid, C22:4(n, 6)cis Docosatetraenoic acid, C22:5(n-3)cis Docosapentaenoic (DPA), C22:5(n-6)cis cis4,7,10,13,16Docosapentaenoic acid, C22:6(n-3)cis Docosahexaenoic (DHA), C24:0 Lignoceric acid, C24:1(n-9)cis Nervonic acid, C25:0 Pentacosanoic acid, C4:0 Butyric acid, C5:0 Valeric acid, C6:0 Caproic acid, C7:0 Heptanoic acid, C8:0 Caprylic acid, C9:0 Nonanoic acid. The method for fatty acid hydrolysis is British Standard 4401 Pt 4:1970. The tests are UKAS accredited to BS ENO/IE17025:2005.The samples were analysed in Eurofins Scientific, an accredited laboratory under ISO:17025.

The results of the average sum of DHA+EPA (mg)/100 g meat in broiler meat are shown in Table 27.

TABLE 27
Sum of DHA + EPA (mg)/100 g meat in broiler meat portions

| | Breast | Thigh |
|---|---|---|
| | +skin | +skin |
| T2 (n = 20) | 75.08 | 194.995 |
| T3 (n = 30) | 52.72 | 60.46 |

### Example 6

### Enrichment of Pork with Omega 3 Fatty Acids

The objective of this research was to determine the best method for omega 3 enrichment of pork. Pigs starting at 60kg (50 days pre-slaughter) and 90kg (25 days pre-slaughter) were balanced for weight, gender (at least 8 gilts in each pen) and assigned into treatment groups. There were 2 pen replicates of each treatment with 15 pigs allocated per pen which were kept in the same group until slaughter. Pigs were weighed at the beginning of the trial and every 4 weeks after. Performance parameters such as feed intake, growth rate and FCR were calculated and pigs were followed to factory so that carcass performance data to include killing-out percentage (KO%) and back fat (P2) could be recorded for pigs on each treatment. Pigs from both start weights were all slaughtered on the same day. In this study 4 dietary treatments were tests over 2 feeding periods to give 8 diets. The four treatments included:

T1 =Control, 25 days

T2 =Control, 50 days

T3 =Premix 1, 25 days

T4 =Premix 1, 50 days

T5 =Premix 2, 25 days

T6 =Premix 2, 50 days

T7 =Premix 3, 25 days

T8 =Premix 3, 50 days

TABLE 28

| | Premix 1 | Premix 2 | Premix 3 |
|---|---|---|---|
| Wheat | 70 | 31 | 59.5 |
| Micronised linseed | 15 | 60 | 20 |
| Linseed oil | 0 | 5 | 0 |
| Dehydrated algal cells | 11 | 0 | 16.5 |
| Antioxidents, emulsifier and | 4 | 4 | 4 |
| anti-caking agent | | | |

On the day slaughter, meat was recovered from gilts as per standard commercial practice. The treatment carcasses were labelled on the loin, shoulder and belly so that samples of each could be obtained and prepared for analysis. The fatty acid profile was analysed from loin meat, loin fat, pork belly, shoulder and rind. The laboratory used was Eurofins Scientific, Dublin. For the loin lean samples, the rind and all visible fat was removed. For the loin fat samples the rind was removed from the fat. The rind samples were taken from the belly. For the belly and shoulder samples the rind was removed.

### Results 50 Days Pre-Slaughter

Pigs on treatment T4 reported a heavier finishing weight compared to the other treatment groups (T2, T6 or T8) although the differences were not statistically significant (Table 29). Numerically all treatments had an increased feed intake compared to pigs within control group with Premix 1 reporting the highest feed intake. Additionally, Premix 1 had the highest growth rate compared to the remaining treatments with Premix 2 and 3 reporting a lower growth rate than pigs on control diet. From 0-28 days pigs within the control treatment had a statistically significant (P<0.05) improved FCR as compared to the remaining three treatment groups. Over 0-50 days numerically pigs on control diet had an improved FCR whereas pigs within treatment Premix 3 reported the worse performance (Table 29).

No statistically significant differences were observed for carcass dead weight, kill out percentage or P2 (Table 30). Numerically Premix 3 reported the highest kill out percentage of 81.13% and back fat of 13.69mm with the control group reporting the lowest kill out percentage and least amount of back fat (Table 30).

TABLE 29
Live performance data for pigs allocated to different
omega 3 enriched diets 50 days pre-slaughter

| Diet | | Control | Premix 1 | Premix 2 | Premix 3 | | |
|---|---|---|---|---|---|---|---|
| No. Pigs | | 29 | 29 | 30 | 29 | S.E.M | Significance |
| Weight | | | | | | | |
| Start | | 62.37 | 63.05 | 63.45 | 63.63 | 0.845 | 0.74 |
| 28 | d | 91.72 | 91.25 | 89.85 | 89.83 | 2.153 | 0.926 |
| 50 | d | 111.88 | 114.34 | 111.68 | 112.27 | 2.439 | 0.858 |
| ADFI (g/d) | | | | | | | |
| 0-28 | d | 2292 | 2496 | 2381 | 2381 | 98.302 | 0.566 |
| 29-50 | d | 2270 | 2746 | 2765 | 2473 | 99.356 | 0.178 |
| 0-50 | d | 2369 | 2606 | 2550 | 2451 | 95.08 | 0.41 |
| ADG (g/d) | | | | | | | |
| 0-28 | d | 1048 | 1007 | 943 | 936 | 51.133 | 0.587 |
| 29-50 | d | 916 | 1049 | 992 | 949 | 57.599 | 0.479 |
| 0-50 | d | 990 | 1026 | 965 | 973 | 33.389 | 0.621 |
| FCR | | | | | | | |
| 0-28 | d | 2.19^{a} | 2.48 ^{b} | 2.53 ^{b} | 2.54 ^{b} | 0.05 | 0.027 |
| 29-50 | d | 2.7 | 2.62 | 2.79 | 2.61 | 0.213 | 0.945 |
| 0-50 | d | 2.39 | 2.54 | 2.64 | 2.52 | 0.064 | 0.191 |

TABLE 30
Carcass performance for pigs allocated to different
omega 3 enriched diets 50 days pre-slaughter

| Diet | Control | Premix 1 | Premix 2 | Premix 3 | S.E.M | Significance |
|---|---|---|---|---|---|---|
| Start Wt | 62.1 | 63.07 | 63.45 | 63.62 | 0.845 | 0.74 |
| End Wt. | 111.83 | 114.33 | 111.68 | 112.29 | 2.438 | 0.909 |
| Dead Wt. | 91.16 | 91.91 | 90.51 | 90.85 | 2.134 | 0.974 |
| KO % | 81 | 78.7 | 81.13 | 79.08 | 1.966 | 0.827 |
| P2 | 12.68 | 13.6 | 13.69 | 12.77 | 0.415 | 0.307 |

### 25 Days Pre-Slaughter

No statistically significant differences were observed in finish weight for pigs within the four treatments. Control pigs had the heaviest finish weight of 115.96 kg whilst treatment Premix 1 reported the lowest finish weight of 112.87 kg but that treatment did have the lightest start weight (Table 31). There was no statistically significant differences in feed intake, growth rate and FCR between treatment groups. Numerically treatment Premix 2 reported the highest feed intake, highest growth rate which was similar to the control treatment and had an improved FCR compared to Premix 1 and Premix 3 treatments. The control treatment did however have the most improved FCR over all treatments with a value of 2.81 (Table 31).

No statistically significant differences were observed in carcass dead weight, kill out percentage and P2 between all four treatments (Table 32). On a numerical basis Premix 3 had the lowest kill out percentage and Premix 2 reported the highest amount of back fat.

TABLE 31
Live performance of pigs allocated to different
omega 3 enriched diets 25 days pre-slaughter

| Diet | | Control | Premix 1 | Premix 2 | Premix 3 | | |
|---|---|---|---|---|---|---|---|
| No. Pigs | | 30 | 28 | 30 | 30 | S.E.M | Significance |
| Weight (kg) | | | | | | | |
| Start | | 92.04 | 90.55 | 93.48 | 91.63 | 1.108 | 0.416 |
| 25 | d | 115.96 | 112.87 | 113.6 | 115.55 | 2.944 | 0.853 |
| ADFI (g/d) | | | | | | | |
| 0-25 | d | 2690 | 2762 | 2567 | 2833 | 72.021 | 0.199 |
| ADG (g/d) | | | | | | | |
| 0-25 | d | 957 | 893 | 805 | 957 | 89.467 | 0.624 |
| FCR | | | | | | | |
| 0-25 | d | 2.81 | 3.09 | 3.19 | 2.96 | 0.317 | 0.751 |

TABLE 32
Carcass performance for pigs allocated to different
omega 3 enriched diets 25 days pre-slaughter

| Diet | Control | Premix 1 | Premix 2 | Premix 3 | S.E.M | Significance |
|---|---|---|---|---|---|---|
| Start Wt | 92.03 | 90.56 | 93.48 | 91.63 | 1.108 | 0.416 |
| End Wt. | 115.93 | 112.96 | 113.6 | 115.55 | 2.944 | 0.853 |
| Dead Wt. | 93.77 | 92.78 | 92.67 | 94.24 | 2.515 | 0.95 |
| KO % | 81.41 | 82.36 | 80.12 | 81.02 | 0.84 | 0.378 |
| P2 | 12.78 | 12.97 | 12.53 | 14.21 | 0.683 | 0.375 |

### Comparison of Performance Data Between Omega 3 Enrichment Over 50 Days and 25 Days Pre Slaughter

Pigs that commenced their omega 3 enrichment 25 days pre-slaughter typically had heavier finish weights upon slaughter. Furthermore, feed intake was higher for pigs that began their treatment diets at 25 days pre-slaughter as opposed to 50 days but generally growth rates were better for pigs on the 50 day treatments with improved FCR's reported for pigs on the 50 day treatments. From comparing 0-25 day enrichment period to the latter period of the 50 day treatment (29-50 day), the 29-50 day enrichment period resulted in numerically higher feed intake for treatment Premix 3, higher growth rates for Premix 1 and Premix 3 and improved FCR's across all regimes (Control, Premix 1,2 and 3).

TABLE 33
Average ALA, EPA and DHA per 100 kcal on pig meat, fat
and rind using various Omega 3 enriched feed treatments

| | Control | T3 | T4 | T5 | T6 | T7 | T8 |
|---|---|---|---|---|---|---|---|
| Averages ALA mg/ | | | | | | | |
| 100 kcal | | | | | | | |
| Loin lean | 108.6 | 90 | 191 | 217.7 | 281.7 | 103.7 | 151.3 |
| Loin fat | 256 | 372 | 461 | 509.7 | 1121.4 | 196.8 | 502.5 |
| Belly | 221.6 | 239 | 339 | 436.7 | 725.8 | 302.7 | 388.9 |
| Shoulder | 213.8 | 280 | 440 | 461.3 | 681 | 298.1 | 390 |
| Rind | 346.8 | 536 | 720 | 588 | 788 | 381.2 | 912 |
| Averages DHA mg/ | | | | | | | |
| 100 kcal | | | | | | | |
| Loin lean | 5.1 | 31 | 56 | 5.9 | 7.4 | 37.2 | 72.2 |
| Loin fat | 6.6 | 65 | 117 | 18.1 | 12.4 | 53.1 | 158 |
| Belly | 5.9 | 46 | 89.6 | 26.2 | 10.1 | 62.2 | 129.8 |
| Shoulder | 10.8 | 50 | 101.7 | 15.6 | 10.7 | 70.2 | 118.7 |
| Rind | 9.4 | 121 | 165.6 | 27 | 12 | 76.8 | 287.2 |
| Averages EPA + | | | | | | | |
| DHA mg/100 kcal | | | | | | | |
| Loin lean | 7.6 | 42 | 70.1 | 11.1 | 24.4 | 46 | 92.3 |
| Loin fat | 9.5 | 74 | 128 | 23.4 | 23.6 | 58.6 | 173.3 |
| Belly | 8.9 | 51 | 103.4 | 32.8 | 24.6 | 70.8 | 143.9 |
| Shoulder | 14 | 58 | 118.4 | 23.1 | 25.2 | 81 | 132.3 |
| Rind | 13.5 | 133 | 188.8 | 35.2 | 33 | 90.4 | 325.9 |

T6 gave the highest concentration of ALA and across all treatments ALA concentrated mostly in the rind. T8 gave the highest concentration of DHA and across all treatments DHA concentrated mostly in the loin fat. T8 gave the highest concentration of EPA and across all treatments DHA concentrated mostly in the rind. ALA, DHA and EPA deposited similarly in the belly and should across all treatments.

No statistically significant differences were found for live performance and carcass performance parameters between all four treatments (Control, Premix 1,2 and 3) across a 25 day and 50 day finishing period. Omega 3 enrichment over a 50 day period has proven to be more beneficial for the performance of animals as opposed to a 25 day pre-slaughter enrichment period.

### Example 7

### Absorption and Accumulation of Chicken Meat Derived Omega-3 PUFAS

A double-blind, controlled, randomized trial with 82 healthy adults was carried out to determine the time course of absorption and accumulation of chicken meat derived Omega-3 PUFAS in humans after eating Omega 3 enriched chicken 3 times per week.

For the trial, 40,000 Ross birds, split housed and sexed, were used. The day old chicks were delivered from commercial hatcheries. They were fed and watered ad libitum with automatic feeders and nipple drinkers. All birds had 23 hours of light per day from day 0 to day 7, and 18 hours of light for the remainder of the crop. The birds were vaccinated at day 18 in accordance with a standard vaccination programme. Birds were fed a standard starter and grower diet (same as control starter and grower diet) (see Table 34) and then offered a finisher ration containing 10% (w/w) of a composition of the invention as defined in Table 35, from 21 days for approximately 20 days (until final kill).

TABLE 34
Standard starter and grower diet, and finisher diet composition.

| | | | Broiler |
|---|---|---|---|
| | Broiler | Broiler | Finisher/ |
| Control diet | Starter | Grower | Withdrawal |
| Maize | 15.0 | 10.0 | 10.0 |
| Wheat | 47.9 | 55.8 | 59.4 |
| Full fat soya | 12.5 | 12.5 | 12.5 |
| Soya bean meal | 19.1 | 15.3 | 11.5 |
| Soyabean oil | 1.35 | 2.64 | 2.89 |
| Calcium carbonate, dicalcium | 2.900 | 2.800 | 2.700 |
| phosphate, sodium bicarbonate, | | | |
| sodium chloride, vitamins and minerals | | | |
| Amino acids | 1.100 | 1.000 | 1.000 |

TABLE 35
Composition of the invention

| Ingredient | % in OmegaPro |
|---|---|
| Wheat | 51.5 |
| Micronised linseed | 30 |
| Dehydrated algae cells | 15 |
| Synthetic/Natural antioxidants | 1.5 |
| Surfactant/emulsifier/binder/flow agent | 2 |

Birds were processed—this typically involves stunning, bleeding, spay washing, de-feathering, scalding, head/foot removal, evisceration, carcass inspection, spray washing, primary chilling, weighing, and secondary chilling. The birds were then portioned as required and frozen until required by the study participants or minced and sent to the laboratory for fatty acid profiling. The average DHA and EPA content of the chicken portions are shown in Table 36.

TABLE 36
DHA and EPA content of chicken portions

| | EPA | DHA | Portion | EPA | DHA |
|---|---|---|---|---|---|
| Food Product | mg/100 g | mg/100 g | size g | mg/portion | mg/portion |
| Control Chicken Skinless Breast | 3.5 | 5 | 120 | 4.2 | 6 |
| Omega-3-PUFA Enriched Chicken | 12 | 65 | 120 | 14 | 78 |
| Skinless Breast | | | | | |
| Control Chicken Skinless Thigh | 9.8 | 11 | 120 | 12 | 13 |
| Omega-3-PUFA Enriched Chicken | 30 | 145 | 120 | 36 | 174 |
| Skinless Thigh | | | | | |

A double-blind, controlled, randomized trial was conducted. Eighty-two healthy participants were randomized to eat at least 3 portions/week of either omega-3-PUFA enriched chicken-meat (n=40) or control chicken-meat (n=42) for 6 months. Each week, each participant family received 1 whole chicken, 4 breasts, 4 thighs, 4 drumsticks, 4 wings plus skin.

Study participants (adults 18 years) were selected according to the following exclusion criteria:1) Previous cardiovascular disease (myocardial infarction, coronary intervention, or stroke), 2) Currently prescribed anti-hypertensive, lipid lowering, or anti-platelet therapy and, 3) Currently regularly taking Omega-3 polyunsaturated fatty acid supplements.

Blood samples were taken at the beginning of the study (Time 0), after 3 Months (T3) and after 6 months (T6) and sent to Mylnefield Lipid Analysis, an external laboratory (Accredited to IS09001:2008). Plasma and red cell levels of C10:0 Capric acid, C10:1(n-1)cis cis-9-Decenoic acid, C12:0 Lauric acid, C12:1(n-1)cis cis-11-Dodecenoic acid, C12:1(n-3)cis cis-9-Dodecenoic acid, C13:0 Tridecanoic acid, C14:0 ante-iso 11-Methyltridecanoic acid, C14:0 iso 12-Methyltridecanoic acid, C14:0 Myristic acid, C14:1(n-5)cis Myristoleic Acid, C15:0 ante-iso 12-Methyltetradecanoic acid, C15:0 iso 13-Methylmyristic acid, C15:0 Pentadecanoic acid, C15:1(n-5)cis cis-10-Pentadecenoic Acid, C16:0 iso 14-Methylpentadecanoic acid, C16:0 Palmitic acid, C16:1(n-5)cis cis-11-Hexadecenoic acid, C16:1(n-7)cis Palmitoleic acid C16:1(n-9)cis cis-5-Hexadecenoic acid, C17:0 ante-iso 14-Methylhexadecanoic acid, C17:0 Heptadecanoic acid, C17:0 iso 15-Methylpalmitic acid, C17:1(n-7)cis cis-10-Heptadecenoic Acid, C18:0 ante-iso 15-Methylheptadecanoic acid, C18:0 iso 16-Methylheptadecanoic acid, C18:0 Stearic acid, C18:1(n-11)trans trans-7-Octadecenoic acid, C18:1(n-6)cis cis-12-Octadecenoic acid, C18:1(n-6)trans trans-12-Octadecenoic Acid, C18:1(n-7)cis cis-Vaccenic Acid, C18:1(n-7)trans trans-Vaccenic acid, C18:1(n-9)cis Oleic acid, C18:1(n-9)trans Elaidic acid, C18:2(n-6)cis Linoleic acid, C18:2(n-6)trans Linolelaidic acid, C18:2conj Total Conjugated Linoleic acid (CLA), C18:3(n-3)cis Alpha-Linolenic acid (ALA), C18:3(n-6)cis Gamma-Linolenic acid (GLA), C18:4(n-3)cis Stearidonic acid, C20:0 Arachidic acid, C20:1(n-11) Gadoleic Acid, C20:1(n9)cis cis-11-Eicosenoic Acid, C20:2(n-6)cis cis-11,14-Ecosadienoic acid, C20:3(n-3)cis cis-11,14,17-Eicosatrienoic acid, C20:3(n-6)cis cis-8,11,14 Eicosatrienoic acid, C20:4(n-3)cis cis-8,11,14,17-Eicosatetraenoic acid, C20:4(n-6)cis Arachidonic Acid, C20:5(n-3)cis Eicosapentenoic acid (EPA), C22:0 Behenic acid, C22:1(n-11)cis Cetoleic acid, C22:1(n-9)cis Erucic acid, C22:2(n-6)cis Docosadienoic acid, C22:4(n, 6)cis Docosatetraenoic acid, C22:5(n-3)cis Docosapentaenoic (DPA), C22:5(n-6)cis cis4,7,10,13,16Docosapentaenoic acid, C22:6(n-3)cis Docosahexaenoic (DHA), C24:0 Lignoceric acid, C24:1(n-9)cis Nervonic acid, C25:0 Pentacosanoic acid, C4:0 Butyri acid, C5:0 Valeric acid, C6:0 Caproic acid, C7:0 Heptanoic acid, C8:0 Caprylic acid, C9:0 Nonanoic acid were measured.

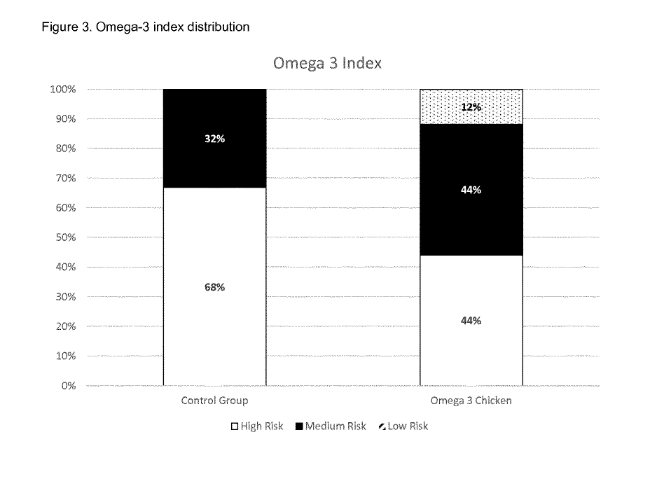

Eating enriched chicken-meat for 6 months resulted in a 16 μg/g (17%, p=0.003) increment in plasma DHA levels. Eating omega-3 enriched chicken-meat resulted in a 9 μg/g (38%, p=0.02) increment in plasma EPA levels. Eating the enriched foods resulted in a substantial shift in the omega-3 index distribution (see FIG. 3). The results of this trial demonstrate that regular consumption of omega-3-PUFA enriched chicken-meat led to an increase in plasma omega-3-PUFA levels, and a considerable reduction in the number of subjects with a high risk omega-3 index. Omega-3-PUFA enriched chicken-meat offers consumers an attractive alternative to eating oily fish or to lifelong taking of supplements, with the potential for substantial human health benefits.

The present invention accordingly provides a composition for animals such as poultry or pigs, based on different sources of omega-3 fatty acids, optionally together with antioxidant(s), flow agent(s) and surfactant(s), to achieve the desired levels of omega-3 fatty acids in the animals. The resulting meat, enriched with Omega-3 fatty acids, provides a range of, for example. poultry and pig meat portions with levels of Omega-3 fatty acids above a threshold limit at which health properties for the consumer can occur. Feeding high levels of Omega-3 fatty acids according to the compositions and methods of the invention allows enrichment of animal meat without detrimental effects on meat sensory quality or animal performance.

1. Animal meat enriched with Omega-3 polyunsaturated fatty acids for use in increasing the Omega-3 polyunsaturated fatty acids levels in human plasma, the use comprising dietary administration of the animal meat enriched with Omega-3 polyunsaturated fatty acids to a subject.

2. Animal meat for use according to claim 1, wherein the animal meat comprises at least 40 mg Omega-3 polyunsaturated fatty acid per 100 g of animal meat.

3. Animal meat for use according to claim 1 or 2, wherein the animal meat comprises 40-1500 mg Omega-3 polyunsaturated fatty acid per 100 g of animal meat.

4. Animal meat for use according to claim 1, wherein the animal meat comprises at least 40 mg Omega-3 polyunsaturated fatty acid per 100 kcal equivalent weight of animal meat.

5. Animal meat for use according to claim 4, wherein the animal meat comprises 40-1500 mg Omega-3 polyunsaturated fatty acid per 100 kcal equivalent weight of animal meat.

6. Animal meat for use according to any one of claims 1-5, wherein Omega-3 polyunsaturated fatty acid is selected from docosahexaenoic acid (DHA), eicosapentaenoic acid (EPA), and a-linolenic acid (ALA).

7. Animal meat for use according to any of claims 1-6, wherein the animal meat is enriched with Omega-3 polyunsaturated fatty acids by dietary administration to the animal of a composition comprising an Omega-3 polyunsaturated fatty acid composition, wherein the composition comprises: (a) 5%-60% algal Omega-3 polyunsaturated fatty acid; and/or (b) 5%-80% linseed Omega-3 polyunsaturated fatty acid.

8. Animal meat for use according to claim 7, wherein the algal Omega-3 polyunsaturated fatty acid is from algal cells selected from any one or more of Chlorella, Spirulina, Schizochytrium, Crypthecodinium, Arthrospira, Porphyridium, Nannochloropsis. a red alga, a brown alga, and a green alga.

9. Animal meat for use according to claim 7 or 8, wherein the algal Omega-3 polyunsaturated fatty acid is from dehydrated algal cells.

10. Animal meat for use according to claim 7, wherein the algal Omega-3 polyunsaturated fatty acid is an algal oil from any one or more of Chlorella, Spirulina, Schizochytrium, Crypthecodinium, Arthrospira, Porphyridium, Nannochloropsis. a red alga, a brown alga, and a green alga.

11. Animal meat for use according to claim 7, wherein the linseed Omega-3 polyunsaturated fatty acid is from milled, ground, or micronized linseed.

12. Animal meat for use according to claim 7, wherein the linseed Omega-3 polyunsaturated fatty acid is from a linseed oil.

13. A method of increasing the Omega-3 polyunsaturated fatty acids levels in human plasma, the method comprising dietary administration of animal meat enriched with Omega-3 polyunsaturated fatty acids to the subject.

14. A method of increasing the Omega-3 polyunsaturated fatty acids levels in human plasma, the method comprising:
(a) enriching animal meat with Omega-3 polyunsaturated fatty acids; and
(b) dietary administration of the animal meat enriched with Omega-3 polyunsaturated fatty acids to the subject.

15. A method according to claim 14, wherein the enriching step (a) comprises dietary administration to the animal of a composition comprising an Omega-3 polyunsaturated fatty acid composition, wherein the composition comprises: (a) 5%-60% algal Omega-3 polyunsaturated fatty acid; and/or (b) 5%-80% linseed Omega-3 polyunsaturated fatty acid.
